(19) United States
(12) Patent Application Publication
(10) Pub. No.: US 2026/0259153 A1
(43) Pub. Date: Sep. 3, 2026

(54) CALIBRATION METHOD AND APPARATUS FOR IMAGING DEVICE AND IMAGING DEVICE

(71) Applicants: NUCTECH COMPANY LIMITED, Beijing (CN); Tsinghua University, Beijing (CN)

(72) Inventors: Li ZHANG, Beijing (CN); Zhiqiang CHEN, Beijing (CN); Yunda SUN, Beijing (CN); Xinbin LI, Beijing (CN); Le SHEN, Beijing (CN); Zhenhua ZHAO, Beijing (CN); Hongkai YANG, Beijing (CN)

(21) Appl. No.: 18/878,613

(22) PCT Filed: Jul. 26, 2023
(86) PCT No.: PCT/CN2023/109291

§ 371 Date: Dec. 23, 2024

(30) Foreign Application Priority Data
Sep. 26, 2022 (CN) 202211177863.7

(51) Int. Cl. G01N 23/046

(52) U.S. Cl. CPC G01N 23/046; G01N 2223/3035; G01N 2223/3307; G01N 2223/401; G01N 2223/419

(57) ABSTRACT

A calibration method and apparatus for an imaging device, and an imaging device are provided. The method includes: calculating a first theoretical intersection line according to a theoretical target point position of a radiation source of the imaging device, a theoretical detection position of a detector, and a phantom position of a calibration phantom; determining first detection projection information for the calibration phantom based on the first theoretical intersection line, an attenuation coefficient of a radiation emitted by the radiation source in the calibration phantom, and a radiation energy spectrum distribution of the radiation source in the imaging device; and adjusting a parameter of the imaging device by using difference information between the first detection projection information and second detection projection information obtained by scanning the calibration phantom with the imaging device, so as to calibrate the imaging device and obtain a calibrated target parameter.

### CROSS-REFERENCE TO RELATED APPLICATION(S)

This application is a Section 371 National Stage Application of International Application No. PCT/CN2023/109291, filed on Jul. 26, 2023, entitled “CALIBRATION METHOD AND APPARATUS FOR IMAGING DEVICE AND IMAGING DEVICE”, which claims priority to Chinese Application No. 202211177863.7, filed on Sep. 26, 2022, the contents of which are incorporated herein by reference in their entireties.

### TECHNICAL FIELD

At least one embodiment of the present disclosure relates to an imaging device, in particular to a calibration method and apparatus for an imaging device, and an imaging device.

### BACKGROUND

In recent years, X-ray computed tomography (CT) technology has been widely used in technical fields such as industrial testing, medical diagnosis, and security inspection. The CT imaging device constructed based on the CT technology may be used to scan objects or human bodies to obtain image information of internal structures of the objects or human bodies. However, due to reasons such as device assembly errors and device aging, geometric errors may occur in components of the CT imaging device such as the X-ray source or the detector, resulting in image defects such as image distortions and ring artifacts in the image information reconstructed based on the CT imaging device, thereby reducing the imaging effect of the image information reconstructed by the CT imaging device.

In related technologies, it is desired to calibrate the device parameter of the CT imaging device to improve the image reconstruction effect of the CT imaging device. However, the calibration efficiency of the relevant device parameter calibration method is low, and there are many image defects in the image information obtained by the calibrated CT imaging device.

### SUMMARY

In view of the above issues, the present disclosure provides a calibration method and apparatus for an imaging device, and an imaging device.

According to a first aspect of the present disclosure, a calibration method for an imaging device is provided, including:
calculating a first theoretical intersection line according to a theoretical target point position of a radiation source of the imaging device, a theoretical detection position of a detector, and a phantom position of a calibration phantom, where the first theoretical intersection line represents a propagation path formed within the calibration phantom after a connecting line between the theoretical target point position and the theoretical detection position passes through the calibration phantom;
determining first detection projection information for the calibration phantom based on the first theoretical intersection line, an attenuation coefficient of a radiation emitted by the radiation source in the calibration phantom, and a radiation energy spectrum distribution of the radiation source in the imaging device; and
adjusting a parameter of the imaging device by using difference information between the first detection projection information and second detection projection information obtained by scanning the calibration phantom with the imaging device, so as to calibrate the imaging device and obtain a calibrated target parameter.

According to the embodiments of the present disclosure, the adjusting a parameter of the imaging device by using difference information between the first detection projection information and second detection projection information obtained by scanning the calibration phantom with the imaging device includes:
processing the difference information between the first detection projection information and the second detection projection information according to a target function to obtain the target parameter, where target difference information between target second detection projection information obtained by scanning the calibration phantom and the first detection projection information converges, wherein the target difference information is obtained after the imaging device is calibrated according to the target parameter.

According to the embodiments of the present disclosure, the target function includes a mapping function and a loss function;
the processing the difference information between the first detection projection information and the second detection projection information according to a target function to obtain the target parameter includes:
processing the first detection projection information and the second detection projection information by using the mapping function, so as to obtain a first projection mapping value and a second projection mapping value respectively;
processing the first projection mapping value and the second projection mapping value by using the loss function, so as to obtain a loss value;
iteratively adjusting the parameter of the imaging device according to the loss value until the loss function converges; and
determining the parameter of the imaging device as the target parameter when the loss function converges.

According to the embodiments of the present disclosure, the imaging device includes a multi-level imaging device, the multi-level imaging device includes L scanning-level imaging apparatuses, and L≥2;
the adjusting a parameter of the imaging device by using difference information between the first detection projection information and second detection projection information obtained by scanning the calibration phantom with the imaging device includes:
adjusting an apparatus imaging parameter of each of the L scanning-level imaging apparatuses based on difference information between the second detection projection information obtained by scanning the calibration phantom with each of the L scanning-level imaging apparatuses and the first detection projection information respectively corresponding to each of the L scanning-level imaging apparatuses, so as to obtain a calibrated target apparatus imaging parameter of each of the L scanning-level imaging apparatuses; and
performing a coordinate system unification on the target apparatus imaging parameter of each of the L scanning-level imaging apparatuses, so as to obtain the calibrated target parameter.

According to the embodiments of the present disclosure, the calibration phantom is provided in N different calibration regions, and N≥2;
the calibration method further includes:
scanning the calibration phantom in each of the N calibration regions by using the imaging device, so as to obtain candidate second detection projection information corresponding to each of the N calibration regions; and
determining the second detection projection information according to the candidate second detection projection information corresponding to each of the N calibration regions.

According to the embodiments of the present disclosure, the calibration phantom is provided in a calibration region, the calibration phantom provided in the calibration region includes M calibration postures, and M≥2;
the calibration method further includes:
scanning the calibration phantom with each of the M calibration postures by using the imaging device, so as to obtain candidate second detection projection information of the calibration phantom corresponding to each of the M calibration postures; and
determining the second detection projection information according to the candidate second detection projection information corresponding to each of the M calibration postures.

According to the embodiments of the present disclosure, the calibration phantom is provided in a scanning region of the imaging device; and
a coverage area of the calibration phantom covering the scanning region is greater than or equal to a preset coverage threshold, and the preset coverage threshold is determined based on a scanning region area of the scanning region.

According to the embodiments of the present disclosure, the calibration phantom includes a calibration phantom unit, and the calibration phantom unit includes a plurality of calibration wires distributed in an array.

According to the embodiments of the present disclosure, the calibration wire includes at least one of: a cylindrical calibration wire, a cubic calibration wire, a conical calibration wire, or a trapezoidal calibration wire.

According to the embodiments of the present disclosure, the parameter of the imaging device includes: a target point position of the radiation source and a detection position of the detector.

According to a second aspect of the present disclosure, a calibration apparatus for an imaging device is provided, including:
a calculation module configured to calculate a first theoretical intersection line according to a theoretical target point position of a radiation source of the imaging device, a theoretical detection position of a detector, and a phantom position of a calibration phantom, where the first theoretical intersection line represents a propagation path formed within the calibration phantom after a connecting line between the theoretical target point position and the theoretical detection position passes through the calibration phantom;
a determination module configured to determine first detection projection information for the calibration phantom based on the first theoretical intersection line, an attenuation coefficient of a radiation emitted by the radiation source in the calibration phantom, and a radiation energy spectrum distribution of the radiation source in the imaging device; and
an adjustment module configured to adjust a parameter of the imaging device by using difference information between the first detection projection information and second detection projection information obtained by scanning the calibration phantom with the imaging device, so as to calibrate the imaging device and obtain a calibrated target parameter.

According to the embodiments of the present disclosure, the adjustment module is further configured to:
process the difference information between the first detection projection information and the second detection projection information according to a target function to obtain the target parameter, where target difference information between target second detection projection information obtained by scanning the calibration phantom and the first detection projection information converges, wherein the target difference information is obtained after the imaging device is calibrated according to the target parameter.

According to the embodiments of the present disclosure, the target function includes a mapping function and a loss function;
processing the difference information between the first detection projection information and the second detection projection information according to the target function to obtain the target parameter includes:
processing the first detection projection information and the second detection projection information by using the mapping function, so as to obtain a first projection mapping value and a second projection mapping value respectively;
processing the first projection mapping value and the second projection mapping value by using the loss function, so as to obtain a loss value;
iteratively adjusting the parameter of the imaging device according to the loss value until the loss function converges; and
determining the parameter of the imaging device as the target parameter when the loss function converges.

According to the embodiments of the present disclosure, the imaging device includes a multi-level imaging device, the multi-level imaging device includes L scanning-level imaging apparatuses, and L≥2;
the adjustment module is further configured to:
adjust an apparatus imaging parameter of each of the L scanning-level imaging apparatuses based on difference information between the second detection projection information obtained by scanning the calibration phantom with each of the L scanning-level imaging apparatuses and the first detection projection information respectively corresponding to each of the L scanning-level imaging apparatuses, so as to obtain a calibrated target apparatus imaging parameter of each of the L scanning-level imaging apparatuses; and
perform a coordinate system unification on the target apparatus imaging parameter of each of the L scanning-level imaging apparatuses, so as to obtain the calibrated target parameter.

According to the embodiments of the present disclosure, the calibration phantom is provided in N different calibration regions, and N≥2;
the calibration apparatus further includes:
a first scanning module configured to scan the calibration phantom in each of the N calibration regions by using the imaging device, so as to obtain second detection projection information corresponding to each of the N calibration regions; and
a first determination module configured to determine the second detection projection information according to the candidate second detection projection information corresponding to each of the N calibration regions.

According to the embodiments of the present disclosure, the calibration phantom is provided in a calibration region, the calibration phantom provided in the calibration region includes M calibration postures;
the calibration apparatus further includes:
a second scanning module configured to scan the calibration phantom with each of the M calibration postures by using the imaging device, so as to obtain second detection projection information of the calibration phantom corresponding to each of the M calibration postures; and
a second determination module configured to determine the second detection projection information according to the candidate second detection projection information corresponding to each of the M calibration postures.

According to the embodiments of the present disclosure, the calibration phantom is provided in a scanning region of the imaging device; and
a coverage area of the calibration phantom covering the scanning region is greater than or equal to a preset coverage threshold, and the preset coverage threshold is determined based on a scanning region area of the scanning region.

According to the embodiments of the present disclosure, the calibration phantom includes a calibration phantom unit, and the calibration phantom unit includes a plurality of calibration wires distributed in an array.

According to the embodiments of the present disclosure, the calibration wire includes at least one of: a cylindrical calibration wire, a cubic calibration wire, a conical calibration wire, or a trapezoidal calibration wire.

According to the embodiments of the present disclosure, the parameter of the imaging device includes: a target point position of the radiation source and a detection position of the detector.

According to a third aspect of the present disclosure, an imaging device is provided, including:
a radiation source configured to emit a radiation beam;
a conveying apparatus configured to convey a detected object, where a conveying channel is defined above the conveying apparatus, and a calibration phantom is provided in the conveying channel;
a detector configured to receive the radiation beam passing through the detected object or the calibration phantom; and
the calibration apparatus mentioned above.

According to the calibration method and apparatus for the imaging device, and the imaging device described in the above embodiments, the parameter of the imaging device may be adjusted according to the difference between the second detection projection information obtained by scanning the calibration phantom with the imaging device and the first detection projection information. In this way, the parameter of the imaging device corresponding to the minimized difference between the second detection projection information and the first detection projection information is determined as the target parameter, thereby completing the calibration of the system parameter of the imaging device and improving the image quality of the reconstructed image output by the CT imaging device.

### BRIEF DESCRIPTION OF THE DRAWINGS

The above content, as well as other purposes, features, and advantages of the present disclosure, will become clearer through the following description of the embodiments of the present disclosure with reference to the accompanying drawings, in which:

FIG. 1 schematically shows an application scenario of a calibration method and apparatus for an imaging device according to an embodiment of the present disclosure;

FIG. 2A schematically shows a flowchart of a calibration method for an imaging device according to an embodiment of the present disclosure;

FIG. 2B schematically shows calibration units and calibration phantoms according to an embodiment of the present disclosure;

FIG. 3A schematically shows a flowchart of processing the difference information between the first detection projection information and the second detection projection information according to the target function to obtain a target parameter according to an embodiment of the present disclosure;

FIG. 3B schematically shows an application scenario of a calibration method for an imaging device according to another embodiment of the present disclosure;

FIG. 3C schematically shows an energy spectrum distribution curve of a radiation source according to an embodiment of the present disclosure;

FIG. 3D schematically shows a variation curve between a mass attenuation coefficient of a calibration phantom and X-ray energy according to an embodiment of the present disclosure;

FIG. 4 schematically shows a flowchart of adjusting a parameter of the imaging device by using difference information between the first detection projection information and second detection projection information obtained by scanning the calibration phantom with the imaging device according to an embodiment of the present disclosure;

FIG. 5 schematically shows a flowchart of adjusting a parameter of the imaging device by using difference information between the first detection projection information and second detection projection information obtained by scanning the calibration phantom with the imaging device according to another embodiment of the present disclosure;

FIG. 6 schematically shows a flowchart of adjusting a parameter of the imaging device by using difference information between the first detection projection information and second detection projection information obtained by scanning the calibration phantom with the imaging device according to another embodiment of the present disclosure;

FIG. 7 schematically shows a block diagram of a structure of a calibration apparatus for an imaging device according to an embodiment of the present disclosure;

FIG. 8 schematically shows an application scenario of an imaging device according to an embodiment of the present disclosure; and

FIG. 9 schematically shows a block diagram of an electronic device suitable for implementing a calibration method for an imaging device according to an embodiment of the present disclosure.

### DETAILED DESCRIPTION OF EMBODIMENTS

Hereinafter, the embodiments of the present disclosure will be described with reference to the drawings. However, it should be understood that these descriptions are only exemplary, and are not intended to limit the scope of the present disclosure. In the following detailed description, for ease of explanation, many specific details are set forth to provide a comprehensive understanding of the embodiments of the present disclosure. However, obviously, one or more embodiments may also be implemented without these specific details. In addition, in the following description, descriptions of well-known structures and technologies are omitted to avoid unnecessarily obscuring the concept of the present disclosure.

The terms used here are only for describing specific embodiments, and are not intended to limit the present disclosure. The terms “include”, “comprise”, etc. used herein indicate an existence of described characteristics, steps, operations and/or components, but do not exclude a presence or addition of one or more other characteristics, steps, operations or components.

Unless otherwise defined, all terms (including technical and scientific terms) used herein have the meanings commonly understood by those skilled in the art. It should be noted that the terms used here should be interpreted as having meanings consistent with the context of the specification, and should not be interpreted in an idealized or overly rigid manner.

In the case of using an expression similar to “at least one of A, B and C, etc.”, generally speaking, it should be interpreted according to the meaning of the expression commonly understood by those skilled in the art (for example, “a system having at least one of A, B, and C” shall include, but is not limited to, a system having A alone, B alone, C alone, A and B, A and C, B and C, and/or A, B and C, etc.).

In the technical solution of the present disclosure, the collection, storage, usage, processing, transmission, provision, disclosure and application of user personal information involved comply with relevant laws and regulations, necessary confidentiality measures have been taken, and do not violate public order and good customs.

In the technical solution of the present disclosure, authorization or consent has been acquired from the user before acquiring or collecting the user personal information.

CT imaging technology is currently widely used in various fields such as medical treatments and security checks. The CT imaging technology may be used to acquire three-dimensional image information of internal structures of detected objects or human bodies without damage. During CT imaging, geometric errors in the device may cause structural distortion in the reconstructed 3D image, resulting in inaccurate CT values or ring artifacts. At present, there are various types of CT devices, including the most common spiral scanning cone beam CT and the static CT at the forefront of research. The former has a simple system structure, so the parameter calibration method is relatively simple, while the latter has a more complex system structure, often requiring simultaneous calibration of a plurality of parameters. The CT imaging system typically consists of three main components: an X-ray machine, an imaging object, and a detector. It is desired to adjust their respective parameters to make them closer to the true values in order to obtain clearer reconstructed images.

The related geometric calibration methods are mainly aimed for spiral scanning cone beam CT devices, such as manually or electrically adjusting the parameters of certain device components on the CT device, relying on subjective judgment or objective indicators to determine the adjustment method of the device components. Considering that the methods of adjusting the device components are usually time-consuming and labor-intensive, and are greatly influenced by subjective factors, it is considered to design a calibration phantom with a specific structure, and then calibrate the corresponding system parameters according to the scanning imaging results of the phantom in the CT device. However, other CT imaging systems, including the static CT device, have more diverse and complex system parameters, and simple geometric calibration methods may not achieve the best image reconstruction effects.

The method of adjusting the component parameters on the CT device based on the image quality of reconstructed images is cumbersome, highly dependent on the professional level of operators, and not suitable for other CT imaging systems with more complex and diverse system structures. This method requires manual judgment of whether the image has reached the optimal level, which not only varies from person to person but is also difficult to be reproducible.

It should be noted that the parameter of the imaging device (or CT device) described in the embodiments of the present disclosure may include geometric parameters of the device in related technologies.

The embodiments of the present disclosure provide a calibration method for an imaging device, including: calculating a first theoretical intersection line according to a theoretical target point position of a radiation source of the imaging device, a theoretical detection position of a detector, and a phantom position of a calibration phantom, where the first theoretical intersection line represents a propagation path formed within the calibration phantom after a connecting line between the theoretical target point position and the theoretical detection position passes through the calibration phantom; determining first detection projection information for the calibration phantom based on the first theoretical intersection line, an attenuation coefficient of a radiation emitted by the radiation source in the calibration phantom, and a radiation energy spectrum distribution of the radiation source in the imaging device; and adjusting a parameter of the imaging device by using difference information between the first detection projection information and second detection projection information obtained by scanning the calibration phantom with the imaging device, so as to calibrate the imaging device and obtain a calibrated target parameter.

According to the embodiments of the present disclosure, by calculating the first theoretical intersection line, which is formed within the calibration phantom after a connecting line between the theoretical target point position and the theoretical detection position passes through the calibration phantom, the theoretical position of the radiation passing through the calibration phantom may be determined when the target point of the radiation source and the detector crystal are located at their respective theoretical positions. Therefore, the first theoretical intersection line, the attenuation coefficient of the radiation in the calibration phantom, and the radiation energy spectrum distribution of the radiation source may be used to calculate the first detection projection information generated when both the target point and the detection crystal are located at the theoretical positions. Then, the parameter of the imaging device may be adjusted according to the difference between the second detection projection information obtained by scanning the calibration phantom with the imaging device and the first detection projection information. Therefore, the parameter of the imaging device corresponding to the minimized difference between the second detection projection information and the first detection projection information may be determined as the target parameter, so as to complete the calibration of the system parameter of the imaging device. In this way, the technical problem of the imaging device calibration process being cumbersome and highly dependent on the professional level of the operator in related technologies may be at least partially solved. Furthermore, the calibration method provided in the embodiments may be repeatedly applied to different CT imaging devices with more complex and diverse system structures. By using the calibration method for the imaging device provided in the embodiments of the present disclosure, accurate calibration of the CT imaging device may be achieved, thereby improving the image quality of the reconstructed images output by the CT imaging device.

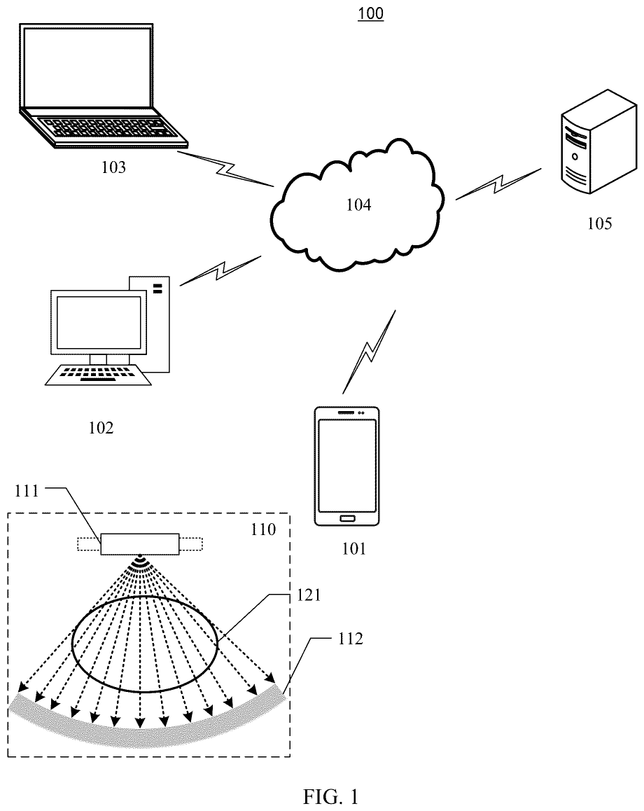

FIG. 1 schematically shows an application scenario of a calibration method and apparatus for an imaging device according to an embodiment of the present disclosure.

As shown in FIG. 1, an application scenario 100 according to this embodiment may include terminal devices 101, 102, 103, a network 104, a server 105, an imaging device 110 and a calibration phantom 121.

The imaging device 110 may include a radiation source 111 and a detector 112, and the calibration phantom 121 is provided between the radiation source 111 and the detector 112.

The network 104 is used to provide a medium for communication links between the terminal devices 101, 102, 103 and the server 105. The network 104 may include various connection types, such as wired/wireless communication links, fiber cables, etc.

The user may use the terminal devices 101, 102, 103 to interact with the server 105 through the network 104 to receive or transmit messages etc. Various communication client applications may be installed on the terminal devices 101, 102, 103, such as shopping applications, web browser applications, search applications, instant messaging tools, email clients, and/or social platform software, etc. (only examples).

The terminal devices 101, 102, 103 may be various electronic devices with a display screen and supporting web browsing, including but not limited to smart phones, tablet computers, laptop computers, and desktop computers, etc.

The server 105 may be a server that provides various services, such as a background management server (only an example) that provides support for websites browsed by the user using the terminal devices 101, 102, and 103. The background management server may analyze and process data such as a received user request, and a processing result (for example, webpage, information, or data acquired or generated according to the user request) is fed back to the terminal device.

It should be noted that the calibration method for the imaging device provided in the embodiments of the present disclosure may generally be executed by the server 105. Accordingly, the calibration apparatus for the imaging device provided by the embodiments of the present disclosure may be generally disposed in the server 105. The calibration method for the imaging device provided by the embodiments of the present disclosure may also be executed by a server or a server cluster different from the server 105 and capable of communicating with the terminal devices 101, 102, 103 and/or the server 105. Accordingly, the calibration apparatus for the imaging device provided by the embodiments of the present disclosure may also be disposed in the server or the server cluster different from the server 105 and capable of communicating with the terminal devices 101, 102, 103 and/or the server 105.

It should be understood that the numbers of terminal devices, networks and servers in FIG. 1 are merely illustrative. According to implementation needs, there may be any number of terminal devices, networks and servers.

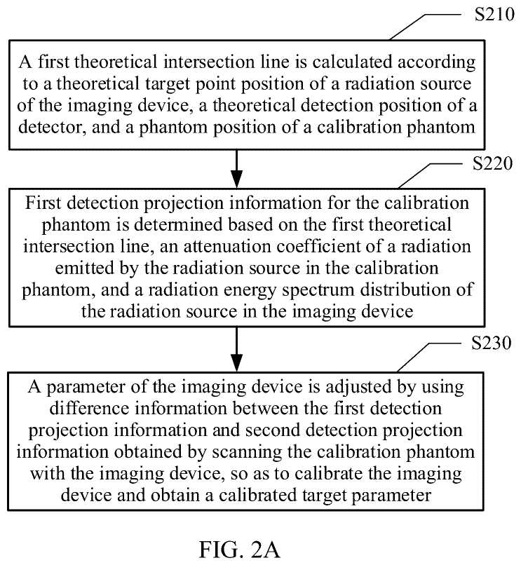

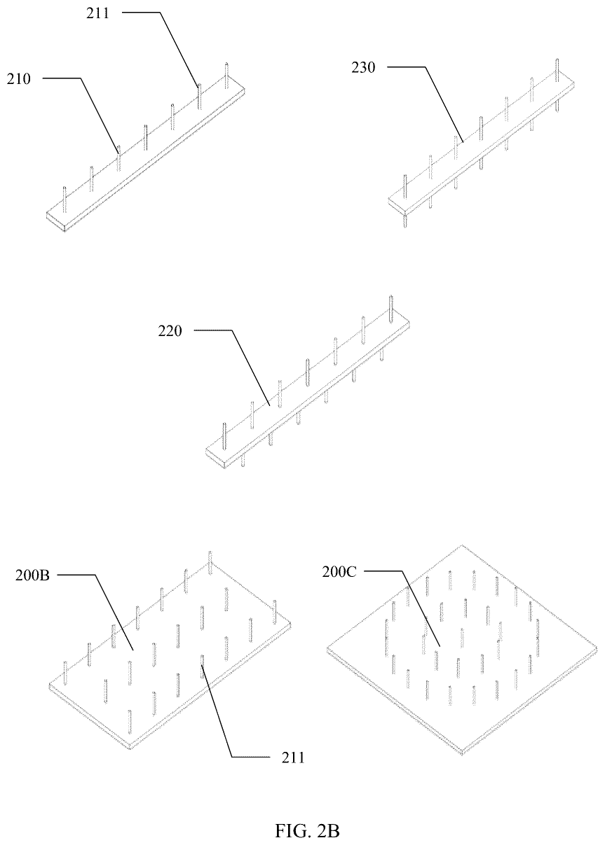

Based on the scenario described in FIG. 1, the calibration method for the imaging device in the embodiments of the present disclosure will be described in detail through FIG. 2A to FIG. 6.

FIG. 2A schematically shows a flowchart of a calibration method for an imaging device according to an embodiment of the present disclosure.

As shown in FIG. 2A, the calibration method for the imaging device in this embodiment includes operations S210 to S230.

In operation S210, a first theoretical intersection line is calculated according to a theoretical target point position of a radiation source of the imaging device, a theoretical detection position of a detector, and a phantom position of a calibration phantom, where the first theoretical intersection line represents a propagation path formed within the calibration phantom after a connecting line between the theoretical target point position and the theoretical detection position passes through the calibration phantom.

In operation S220, first detection projection information for the calibration phantom is determined based on the first theoretical intersection line, an attenuation coefficient of a radiation emitted by the radiation source in the calibration phantom, and a radiation energy spectrum distribution of the radiation source in the imaging device.

In operation S230, a parameter of the imaging device is adjusted by using difference information between the first detection projection information and second detection projection information obtained by scanning the calibration phantom with the imaging device, so as to calibrate the imaging device and obtain a calibrated target parameter.

According to the embodiments of the present disclosure, the imaging device may include, for example, a CT imaging device. In this embodiment, the radiation source of the imaging device emits radiation through a target point, and the radiation passes through the calibration phantom and is received by the detector. The detector may convert the received radiation signal into an electrical signal, and then the electrical signal may be used to construct a detection image of the calibration phantom.

It should be noted that there is no limitation on the number of radiation sources and/or detectors of the imaging device in the embodiments of the present disclosure. For example, the number of radiation sources and the number of detectors may be one, or may also be more.

According to the embodiments of the present disclosure, the theoretical target point position and the theoretical detection position of the detector may be positions where imaging effect of a detection image generated by the imaging device is optimal. For example, the theoretical detection position of the detector may be a theoretical position of a detection crystal receiving the radiation signal. However, a displacement of components of the imaging device may be caused due to reasons such as device errors and abnormal movements, resulting in a decrease in the image quality of an actual detection image obtained by scanning a detection object with the imaging device.

According to the embodiments of the present disclosure, the first theoretical intersection line may be a coordinate position of the first theoretical intersection line in a coordinate system of the imaging device. A linear attenuation coefficient of the calibration phantom may be determined by a material constructing the calibration phantom. The phantom position of the calibration phantom may be a coordinate position of the calibration phantom in the coordinate system corresponding to the imaging device, such as a coordinate position of each granularity unit of the calibration phantom.

It should be noted that the material of the calibration phantom may be an elemental material (such as aluminum), a single compound (such as PMMA), or a mixture (such as ceramics, etc.). The material of the calibration phantom is not limited in the embodiments of the present disclosure, as long as the attenuation coefficient of the radiation in the calibration phantom may be obtained based on the material of the calibration phantom.

According to the embodiments of the present disclosure, the attenuation coefficient may include a parameter used to characterize the attenuation of the radiation in the calibration phantom in related technologies, such as a linear attenuation coefficient or a mass attenuation coefficient. The specific type of the attenuation coefficient is not limited in the embodiments of the present disclosure, and those skilled in the art may select the attenuation coefficient according to actual needs.

According to the embodiments of the present disclosure, the radiation energy spectrum distribution of the radiation source in the imaging device may include a radiation energy spectrum distribution curve, which may be obtained through measurement methods in related technologies. For example, assuming that the energy spectrum distribution of the same radiation source remains consistent under the same operating voltage condition, the radiation energy spectrum distribution curve of the radiation source under the operating voltage condition may be obtained through measurement.

It should be noted that the radiation energy spectrum distribution may include a radiation energy spectrum distribution curve. However, the present disclosure is not limited to this. The radiation energy spectrum distribution may also include a radiation energy spectrum distribution point graph, etc. The specific type of the radiation energy spectrum distribution is not limited in the embodiments of the present disclosure. Those skilled in the art may design it according to actual needs, as long as it may characterize the distribution of radiation frequency energy.

According to the embodiments of the present disclosure, by calculating the first theoretical intersection line, which is formed within the calibration phantom after a connecting line between the theoretical target point position and the theoretical detection position passes through the calibration phantom, the theoretical position of the radiation passing through the calibration phantom may be determined when the target point of the radiation source and the detector crystal are located at their respective theoretical positions. Therefore, the first theoretical intersection line, the attenuation coefficient of the radiation in the calibration phantom, and the radiation energy spectrum distribution of the radiation source may be used to calculate the first detection projection information generated when both the target point and the detection crystal are located at the theoretical positions. Then, the parameter of the imaging device may be adjusted according to the difference between the second detection projection information obtained by scanning the calibration phantom with the imaging device and the first detection projection information. Therefore, the parameter of the imaging device corresponding to the minimized difference between the second detection projection information and the first detection projection information may be determined as the target parameter, so as to complete the calibration of the system parameter of the imaging device. In this way, the technical problem of the imaging device calibration process being cumbersome and highly dependent on the professional level of the operator in related technologies may be at least partially solved. Furthermore, the calibration method provided in this embodiment may be repeatedly applied to different CT imaging devices with more complex and diverse system structures. By using the calibration method for the imaging device provided in the embodiments of the present disclosure, accurate calibration of the CT imaging device may be achieved, thereby improving the image quality of the reconstructed images output by the CT imaging device.

According to the embodiments of the present disclosure, the parameter of the imaging device may include: a target point position of the radiation source and a detection position of the detector.

According to the embodiments of the present disclosure, the target point position of the radiation source and the detection position of the detector may be actual coordinate positions of the target point of the radiation source and the detector after the imaging device is assembled. Due to assembly errors and other reasons, the actual coordinate positions may deviate from the theoretical positions, and the offset direction and distance of each actual coordinate position are difficult to be measured. Through the calibration method provided by the embodiments of the present disclosure, the difference information between the first detection projection information and the second detection projection information may be used as the optimization target, and at least the target point position of the radiation source and the detection position of the detector may be adjusted, so that the target point position and the detection position corresponding to the minimized difference information are determined as the target parameters of the imaging device, thereby achieving the calibration of the imaging device, at least partially solving the technical problem of difficulty in calibrating the imaging device caused by assembly errors and abnormal movements, improving the calibration accuracy and calibration speed of the imaging device, and achieving an improvement in the imaging effect of the imaging device.

According to the embodiments of the present disclosure, the calibration phantom is provided in a scanning region of the imaging device; and a coverage area of the calibration phantom covering the scanning region is greater than or equal to a preset coverage threshold, where the preset coverage threshold is determined based on a scanning region area of the scanning region.

According to the embodiments of the present disclosure, the calibration phantom may be provided as a whole in the scanning region, and the coverage area of the calibration phantom covering the scanning region may be a coverage area of a shape formed by the outermost edge of the calibration phantom covering the scanning region. When the coverage area of the calibration phantom covering the scanning region is greater than or equal to the preset coverage threshold, for example, when the outermost edge of the calibration phantom coincides with the boundary of the scanning region and the calibration phantom is provided as a whole in the scanning region, the imaging device may scan the calibration phantom completely, thereby achieving the optimal imaging effect of the reconstructed detection image generated after scanning the calibration phantom with the imaging device, and avoiding the degradation of image quality in local regions of the detection image.

It should be noted that the shape and structure of the calibration phantom are not limited in the embodiments of the present disclosure. For example, the calibration phantom may have an irregular shape. However, the present disclosure is not limited to this, the calibration phantom may have a regular shape, such as a cube, etc. Those skilled in the art may select the shape and structure of the calibration phantom according to the actual situation.

According to the embodiments of the present disclosure, the calibration phantom includes a calibration phantom unit, and the calibration phantom unit includes a plurality of calibration wires distributed in an array.

According to the embodiments of the present disclosure, the calibration wire includes at least one of: a cylindrical calibration wire, a cubic calibration wire, a conical calibration wire, or a trapezoidal calibration wire.

FIG. 2B schematically shows calibration units and calibration phantoms according to an embodiment of the present disclosure.

As shown in FIG. 2B, each of the calibration phantom units 210, 220, and 230 may include a plurality of calibration wires 211 distributed in an array. The calibration wires 211 in the calibration phantom units 210, 220, and 230 may have different fixing methods, that is, the calibration wires may be constructed into calibration phantom units according to different array distributions.

Furthermore, the calibration phantom unit 210 may be included in the calibration phantom 200B, so that the calibration phantom 200B includes calibration wires 211 arranged in a matrix. However, the present disclosure is not limited to this. The calibration phantom 200C may also include calibration wires distributed in a circular array.

It should be noted that the shape, structure, and arrangement of the calibration phantom, the calibration unit, or the calibration wire in FIG. 2B are all exemplary embodiments and do not limit the specific structural form of the calibration phantom. Those skilled in the art may design diversified calibration phantoms according to actual needs, such as designing calibration phantoms through the diversification of the arrangement of calibration wires.

According to the embodiments of the present disclosure, the calibration phantom with the regular shape and the regular form may be obtained by providing the calibration phantom with a plurality of calibration wires distributed in an array, which may improve the imaging effect of the detection image obtained after scanning the calibration phantom with the imaging device. This may improve the accuracy and reliability of the second detection position information, thereby improving the accuracy of the target parameter generated later and enhancing the detection effect of the calibrated imaging device.

According to the embodiments of the present disclosure, in operation S230, the adjusting a parameter of the imaging device by using difference information between the first detection projection information and second detection projection information obtained by scanning the calibration phantom with the imaging device includes:

processing the difference information between the first detection projection information and the second detection projection information according to a target function to obtain the target parameter, where target difference information between target second detection projection information obtained by scanning the calibration phantom and the first detection projection information converges, where the target difference information is obtained after the imaging device is calibrated according to the target parameter.

According to the embodiments of the present disclosure, the target function may include algorithms used in related technologies for fitting the difference information, such as least squares functions. The specific algorithm type of the target function is not limited in the embodiments of the present disclosure, as long as it may be used to determine that the target difference information between the target second detection projection information and the first detection projection information converges.

According to the embodiments of the present disclosure, the target function includes a mapping function and a loss function.

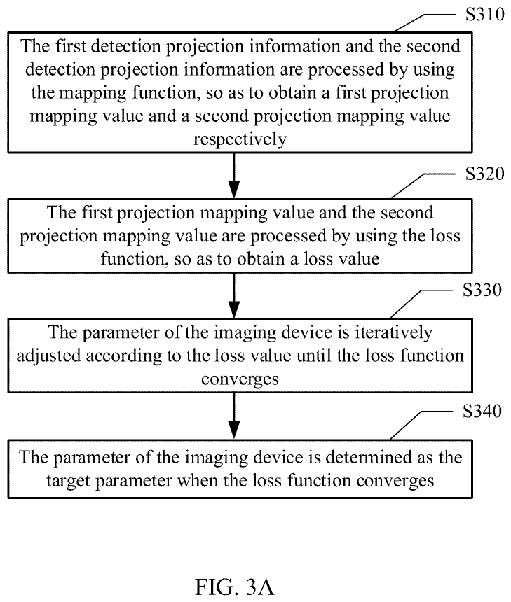

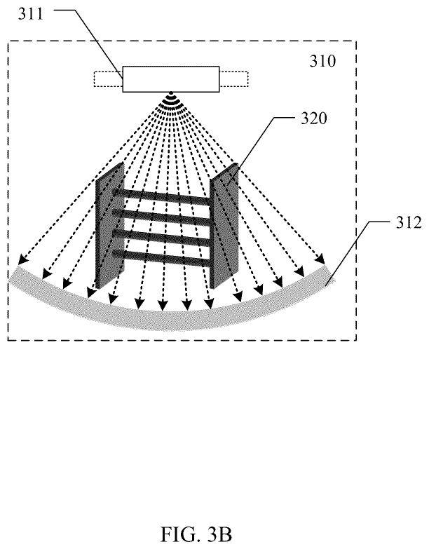

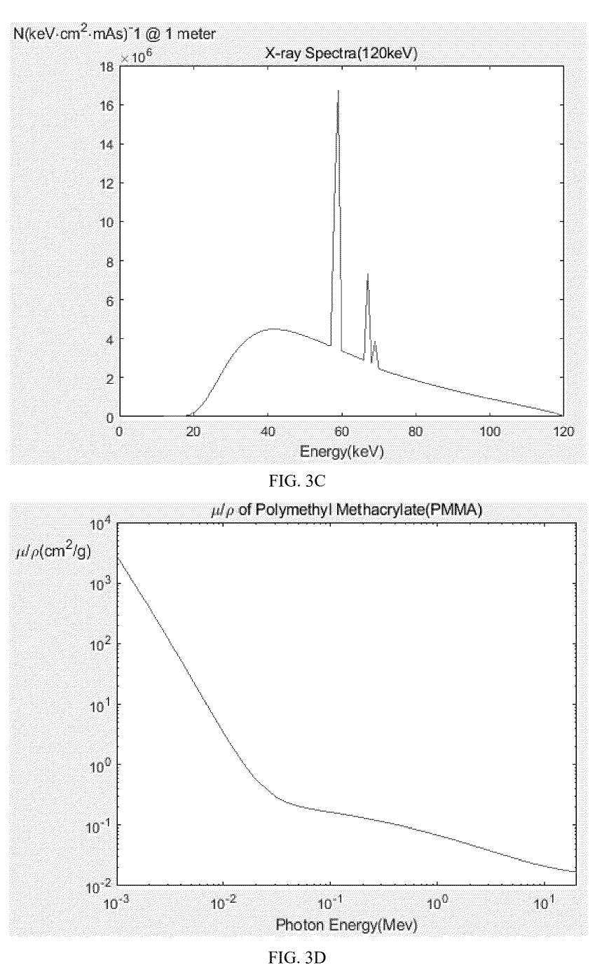

FIG. 3A schematically shows a flowchart of processing the difference information between the first detection projection information and the second detection projection information according to the target function to obtain a target parameter according to an embodiment of the present disclosure.

As shown in FIG. 3A, in this embodiment, the processing the difference information between the first detection projection information and the second detection projection information according to a target function to obtain the target parameter includes operations S310 to S340.

In operation S310, the first detection projection information and the second detection projection information are processed by using the mapping function, so as to obtain a first projection mapping value and a second projection mapping value respectively.

In operation S320, the first projection mapping value and the second projection mapping value are processed by using the loss function, so as to obtain a loss value.

In operation S330, the parameter of the imaging device is iteratively adjusted according to the loss value until the loss function converges.

In operation S340, the parameter of the imaging device is determined as the target parameter when the loss function converges.

According to the embodiments of the present disclosure, the first detection projection information and the second detection projection information may be used as independent variables of the mapping function, that is, the mapping function may be represented as f( ), and the first projection mapping value and the second projection mapping value may be represented as f(P1) and f(P2), respectively. The difference (i.e., the loss value) between the first projection mapping value and the second projection mapping value is used as the optimization target, the loss value is processed using the loss function to iteratively adjust the parameter of the imaging device until the loss function converges, and the parameter corresponding to the converged loss function is determined as the target parameter, thereby achieving calibration of the imaging device.

According to the embodiments of the present disclosure, the minimum difference between the target parameter of the imaging device and the theoretical position of the imaging device may be determined by using the difference (i.e., the loss value) between the first projection mapping value and the second projection mapping value as the optimization target, thereby improving the generalization ability of the calibration method, enabling the calibration method to adapt to the imaging device with complex components, and enhancing the calibration efficiency of the imaging device.

It should be noted that the specific number of times to iteratively adjust the parameter of the imaging device may be one or multiple times. In the embodiments of the present disclosure, there is no limitation of the specific number of times to adjust the parameter of the imaging device.

FIG. 3B schematically shows an application scenario of a calibration method for an imaging device according to another embodiment of the present disclosure.

FIG. 3C schematically shows an energy spectrum distribution curve of a radiation source according to an embodiment of the present disclosure.

FIG. 3D schematically shows a variation curve between a mass attenuation coefficient of a calibration phantom and X-ray energy according to an embodiment of the present disclosure.

As shown in FIG. 3B, FIG. 3C, and FIG. 3D, the application scenario may include an imaging device 310 and a calibration phantom 320. The imaging device 310 may include a radiation source 311 and a detector 312. A target point of the radiation source 311 may emit a radiation signal to the calibration phantom 320, which may be X-rays. After passing through the calibration phantom 320, the radiation signal may be received by the detector 312, so as to obtain the second detection projection information P2 obtained by scanning the calibration phantom 320 with the imaging device 310.

According to the embodiments of the present disclosure, it may be assumed that the energy spectrum distribution of the radiation signal generated by radiation source 311 remains consistent under the same operating voltage condition. For example, the X-ray signal generated by the radiation source 311 at an operating voltage of 120 kVp may be represented by the energy spectrum distribution curve shown in FIG. 3C.

According to the embodiments of the present disclosure, when a material of the calibration phantom 320 is a PMMA material, the linear attenuation coefficient μ of the calibration phantom 320 may be determined, and the variation curve of the mass attenuation coefficient μ/ρ of the calibration phantom 320 and the energy of the X-ray signal generated by the radiation source 311 may be represented by FIG. 3D. ρ represents a density of the cylindrical calibration wire in the calibration phantom 320.

After obtaining the radiation energy spectrum distribution curve of the radiation source 311 and the mass attenuation coefficient of the calibration phantom 320, the theoretical spatial three-dimensional coordinate of the first theoretical intersection line may be calculated according to the theoretical target point position of the radiation source 311, the theoretical detection position of the detector 312, and the phantom position of the calibration phantom (that is, in the coordinate system of the imaging device 310, the theoretical spatial three-dimensional coordinate of the target point of the radiation source 311, the theoretical spatial three-dimensional coordinate of the detector pixel of the detector 312, and the spatial three-dimensional coordinate of the calibration phantom 320), so as to determine the propagation path of the X-ray beam within the calibration phantom 320.

Then, the first detection projection information P1 may be calculated by using equation (1).

$$P 1 = \frac{I}{{I}_{0}} = \int S ( E ) exp [ - {\int }_{L} [ \frac{μ ( E , l )}{ρ} ] ρ dl ] dE ; \qquad ( 1 )$$

In the equation (1), I₀ represents a radiation incident intensity of the radiation signal reaching a surface of the calibration phantom, I represents a radiation emission intensity of the radiation signal passing through the calibration phantom, E represents energy of the radiation signal, ρ represents a density of the calibration phantom, L represents a length of the first theoretical intersection line, and l represents a length of the material corresponding to the density ρ in the length of the first theoretical intersection line.

It should be understood that in the case where the material of the calibration phantom 320 is a uniform PMMA material, (E, l)/ρ may be simplified as μ(E)/ρ, and the equation (1) may be further simplified as equation (2).

$$P 1 = \int S ( E ) exp [ - {\int }_{L} [ \frac{μ ( E )}{ρ} ] ρ dl ] dE ; \qquad ( 2 )$$

After obtaining the first detection projection information P1 and the second detection projection information P2, a difference between the first detection projection information P1 and the second detection projection information P2 may be used as an optimization target. The target parameter of the imaging device 310 may be obtained by adjusting the parameter of the imaging device 310 to minimize such difference. For example, the target parameter may be determined by using the method described in the above embodiment, thereby achieving calibration of the imaging device 310.

According to the embodiments of the present disclosure, in a case where the material of the calibration phantom is a uniform material, the linear attenuation coefficient of the material may be used to calculate the first detection projection information P1; in a case where the calibration phantom is constructed by different materials, the mass attenuation coefficient of the material may be used to calculate the first detection projection information P1, thereby further improving the adaptability and generalization ability of the calibration method provided by the embodiments of the present disclosure for calibrating the imaging device by using different calibration phantoms.

It should be understood that the imaging device may scan the calibration phantom according to a preset scanning angle interval. The scanning angle interval may be designed according to actual needs. After scanning the entire scanning region according to the preset scanning angle interval, the imaging device may complete one scan to obtain complete second detection projection information for the calibration phantom. That is, there may be a plurality of second detection projection information, and each second detection projection information is associated with an actual coordinate position of a detector crystal receiving the radiation signal.

It should be noted that in a case where the imaging device 310 in FIG. 3B is a ring CT imaging device (also known as a ring CT system) in the related technologies, the radiation source and the detector are relatively stationary and rotate around the calibration phantom for scanning. In order to calibrate the imaging device more accurately, the second detection projection information obtained from multi-view scanning and the first detection projection information corresponding to each angle of view may be used to increase the data volume for the target parameter, thereby improving the calibration effect of the imaging device. In order to achieve better calibration results, the scanning angle interval may be further reduced to increase the number of scanning angles, thereby expanding the data volume and laying the foundation for generating the target parameter accurately in the future.

According to the embodiments of the present disclosure, the imaging device includes a multi-level imaging device, the multi-level imaging device includes L scanning-level imaging apparatuses, and L≥2.

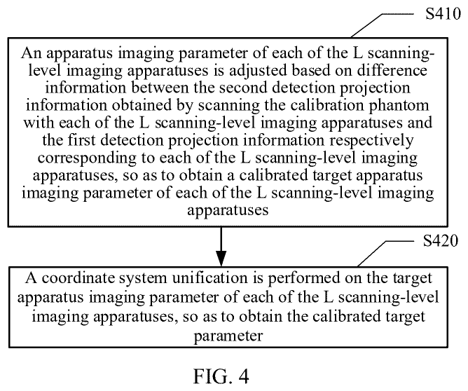

FIG. 4 schematically shows a flowchart of adjusting a parameter of the imaging device by using difference information between the first detection projection information and second detection projection information obtained by scanning the calibration phantom with the imaging device according to an embodiment of the present disclosure.

As shown in FIG. 4, in this embodiment, the adjusting a parameter of the imaging device by using difference information between the first detection projection information and second detection projection information obtained by scanning the calibration phantom with the imaging device includes operations S410 to S420.

In operation S410, an apparatus imaging parameter of each of the L scanning-level imaging apparatuses is adjusted based on difference information between the second detection projection information obtained by scanning the calibration phantom with each of the L scanning-level imaging apparatuses and the first detection projection information respectively corresponding to each of the L scanning-level imaging apparatuses, so as to obtain a calibrated target apparatus imaging parameter of each of the L scanning-level imaging apparatuses.

In operation S420, a coordinate system unification is performed on the target apparatus imaging parameter of each of the L scanning-level imaging apparatuses, so as to obtain the calibrated target parameter.

According to the embodiments of the present disclosure, each scanning-level imaging apparatus may include a radiation source and a detector, so as to constitute one scanning level of the imaging device. The imaging device may construct a detection image for the detected object according to the projection information obtained by scanning the detected object with each of the L scanning-level imaging apparatuses.

According to the embodiments of the present disclosure, the target apparatus imaging parameter corresponding to each scanning-level imaging apparatus may be obtained based on the coordinate system of each scanning-level imaging apparatus. The coordinate system unification is performed on the target apparatus imaging parameter of each of the L scanning-level imaging apparatuses. For example, the coordinate system of each of the L scanning-level imaging apparatuses may be unified as the coordinate system of one of the L scanning-level imaging apparatus, so that the coordinate system unification may be achieved on the target apparatus imaging parameters corresponding to the L scanning-level imaging apparatuses. In this way, the imaging device with complex components may be conveniently calibrated, thereby improving the calibration efficiency of the imaging device.

According to the embodiments of the present disclosure, the calibration phantom is provided in N different calibration regions, and N≥2.

The calibration method may further include: scanning the calibration phantom in each of the N calibration regions by using the imaging device, so as to obtain candidate second detection projection information corresponding to each of the N calibration regions; and determining the second detection projection information according to the candidate second detection projection information corresponding to each of the N calibration regions.

According to the embodiments of the present disclosure, the second detection projection information may include respective candidate second detection projection information corresponding to each of the N calibration regions, and the candidate second detection projection information respectively corresponding to each of the N calibration regions may also have a region identifier representing the corresponding calibration region, thereby facilitating the differentiation of the calibration region corresponding to the second detection projection information.

According to the embodiments of the present disclosure, by providing the calibration phantom in N different calibration regions and obtaining the first and second detection projection information corresponding to each calibration region, on the one hand, the data volume used to obtain the target parameter may be increased, and on the other hand, the imaging effect of the calibrated imaging device for the detection image in each calibration region may be improved, thereby improving the imaging effect of the detection images reconstructed by the imaging device in a plurality of calibration regions, achieving overall optimization of the imaging effect of the imaging device, and avoiding a decrease in the scanning imaging effect of the imaging device for the detected objects provided in some calibration regions.

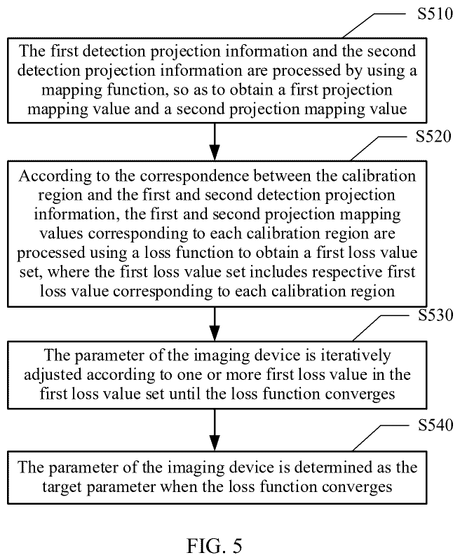

FIG. 5 schematically shows a flowchart of adjusting a parameter of the imaging device by using difference information between the first detection projection information and second detection projection information obtained by scanning the calibration phantom with the imaging device according to another embodiment of the present disclosure.

As shown in FIG. 5, in this embodiment, adjusting a parameter of the imaging device by using difference information between the first detection projection information and second detection projection information obtained by scanning the calibration phantom with the imaging device may include operations S510 to S540.

In operation S510, the first detection projection information and the second detection projection information are processed by using a mapping function, so as to obtain a first projection mapping value and a second projection mapping value, where the first detection projection information and the second detection projection information correspond to the same calibration region.

In operation S520, according to the correspondence between the calibration region and the first and second detection projection information, the first and second projection mapping values corresponding to each calibration region are processed using a loss function to obtain a first loss value set, where the first loss value set includes respective first loss value corresponding to each calibration region.

In operation S530, the parameter of the imaging device is iteratively adjusted according to one or more first loss values in the first loss value set until the loss function converges.

In operation S540, the parameter of the imaging device is determined as the target parameter when the loss function converges.

According to the embodiments of the present disclosure, the target parameter may be determined based on gradient descent algorithm to reduce the difference between the target parameter and the theoretical parameter, thereby improving the imaging accuracy of the calibrated imaging device.

According to the embodiments of the present disclosure, the calibration phantom is provided in a calibration region, and the calibration phantom provided in the calibration region includes M calibration postures.

The calibration method for the imaging device may further include: scanning the calibration phantom with each of the M calibration postures by using the imaging device, so as to obtain candidate second detection projection information of the calibration phantom corresponding to each of the M calibration postures; and determining the second detection projection information according to the candidate second detection projection information corresponding to each of the M calibration postures.

According to the embodiments of the present disclosure, the calibration posture may include a method of setting the calibration phantom in the calibration region. Different calibration postures of the calibration phantom may be achieved through flipping, moving, rotating, and the like.

According to the embodiments of the present disclosure, the second detection projection information may include candidate second detection projection information corresponding to each of M calibration postures, thereby expanding the data volume of the second detection projection information.

According to the embodiments of the present disclosure, a plurality of calibration postures may be provided for the calibration phantom in the calibration region that needs to improve the image reconstruction effect, thereby increasing the data volume of the first and second detection projection information for the calibration region, and enhancing the imaging effect of the calibrated imaging device for the calibration region.

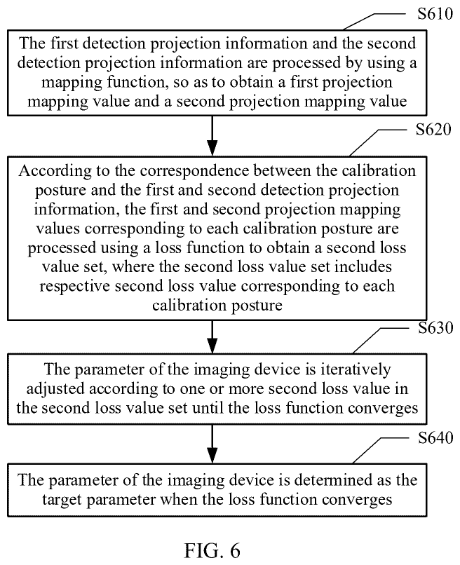

FIG. 6 schematically shows a flowchart of adjusting a parameter of the imaging device by using difference information between the first detection projection information and second detection projection information obtained by scanning the calibration phantom with the imaging device according to another embodiment of the present disclosure.

As shown in FIG. 6, in this embodiment, adjusting a parameter of the imaging device by using difference information between the first detection projection information and second detection projection information obtained by scanning the calibration phantom with the imaging device may include operations S610 to S640.

In operation S610, the first detection projection information and the second detection projection information are processed by using a mapping function, so as to obtain a first projection mapping value and a second projection mapping value, where the first detection projection information and the second detection projection information correspond to the same calibration posture.

In operation S620, according to the correspondence between the calibration posture and the first and second detection projection information, the first and second projection mapping values corresponding to each calibration posture are processed using a loss function to obtain a second loss value set, where the second loss value set includes respective second loss value corresponding to each calibration posture.

In operation S630, the parameter of the imaging device is iteratively adjusted according to one or more second loss values in the second loss value set until the loss function converges.

In operation S640, the parameter of the imaging device is determined as the target parameter when the loss function converges.

It should be noted that the first loss value and the second loss value in the embodiments of the present disclosure are only for the purpose of distinguishing and explaining the correspondence of the loss values, and do not limit that the first loss value corresponds to the calibration region, nor do they limit that the second loss value corresponds to the calibration posture. Different calibration postures may be provided for the calibration phantom in different calibration regions to obtain the loss value corresponding to the calibration phantom with the calibration posture in the calibration region. The loss value may be the first loss value or the second loss value, which is not limited in the embodiments of the present disclosure.

According to the embodiments of the present disclosure, multi-view scanning may also be performed on the calibration phantom provided in the calibration region, so as to obtain the first and second detection projection information corresponding to each angle of view. Then, the target parameter may be determined according to the difference between respective first detection projection information and respective second detection projection information corresponding to each angle of view, so as to complete the calibration of the imaging device.

It should be understood that the second detection projection information corresponding to each scanning angle of view obtained by scanning the calibration phantom from a plurality of angle of views is similar to scanning the calibration phantom with a plurality of calibration postures from a single angle of view, i.e. the calibration phantom may be scanned from different relative angles.

Based on the calibration method for the imaging device mentioned above, the present disclosure further provides a calibration apparatus for the imaging device. The apparatus will be described in detail below in conjunction with FIG. 8.

According to the embodiments of the present disclosure, the calibration method for the imaging device provided in the embodiments of the present disclosure may be used to quickly, effectively, and accurately obtain the optimal system parameter of the imaging device, i.e. the target parameter, so that the calibrated imaging device may reconstruct and obtain the detection image with better image effects. Besides, the calibration method for the imaging device provided in the embodiments of the present disclosure may also cooperate with other modules in the imaging device to more accurately carry out works such as object recognition, achieving better practical application effects.

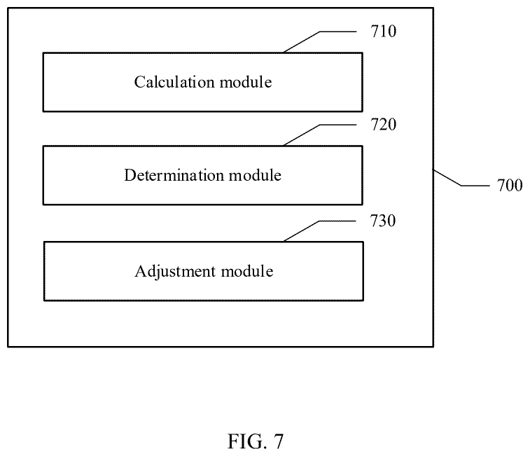

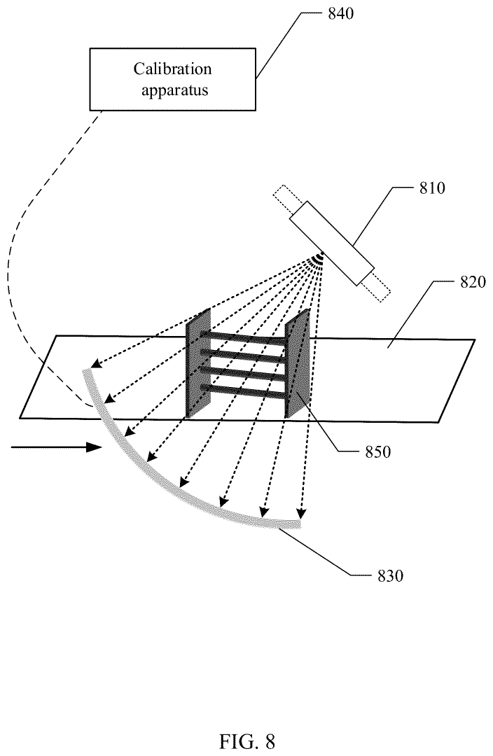

FIG. 7 schematically shows a block diagram of a structure of a calibration apparatus for an imaging device according to an embodiment of the present disclosure.

As shown in FIG. 7, in this embodiment, a calibration apparatus 700 for an imaging device includes a calculation module 710, a determination module 720 and an adjustment module 730.

The calculation module 710 is used to calculate a first theoretical intersection line according to a theoretical target point position of a radiation source of the imaging device, a theoretical detection position of a detector, and a phantom position of a calibration phantom, where the first theoretical intersection line represents a propagation path formed within the calibration phantom after a connecting line between the theoretical target point position and the theoretical detection position passes through the calibration phantom.

The determination module 720 is used to determine first detection projection information for the calibration phantom based on the first theoretical intersection line, an attenuation coefficient of a radiation emitted by the radiation source in the calibration phantom, and a radiation energy spectrum distribution of the radiation source in the imaging device.

The adjustment module 730 is used to adjust a parameter of the imaging device by using difference information between the first detection projection information and second detection projection information obtained by scanning the calibration phantom with the imaging device, so as to calibrate the imaging device and obtain a calibrated target parameter.

According to the embodiments of the present disclosure, the adjustment module is further used to: process the difference information between the first detection projection information and the second detection projection information according to a target function to obtain the target parameter, where target difference information between target second detection projection information obtained by scanning the calibration phantom and the first detection projection information converges, wherein the target difference information is obtained after the imaging device is calibrated according to the target parameter.

According to the embodiments of the present disclosure, the target function includes a mapping function and a loss function.

Processing the difference information between the first detection projection information and the second detection projection information according to the target function to obtain the target parameter includes: processing the first detection projection information and the second detection projection information by using the mapping function, so as to obtain a first projection mapping value and a second projection mapping value respectively; processing the first projection mapping value and the second projection mapping value by using the loss function, so as to obtain a loss value; iteratively adjusting the parameter of the imaging device according to the loss value until the loss function converges; and determining the parameter of the imaging device as the target parameter when the loss function converges.

According to the embodiments of the present disclosure, the adjustment module may include a first processing sub-module, a second processing sub-module, a first adjustment sub-module, and a first determination sub-module.

The first processing sub-module is used to process the first detection projection information and the second detection projection information by using the mapping function, so as to obtain a first projection mapping value and a second projection mapping value respectively.

The second processing sub-module is used to process the first projection mapping value and the second projection mapping value by using the loss function, so as to obtain a loss value.

The first adjustment sub-module is used to iteratively adjust the parameter of the imaging device according to the loss value until the loss function converges. The first determination sub-module is used to determine the parameter of the imaging device as the target parameter when the loss function converges.

According to the embodiments of the present disclosure, the imaging device includes a multi-level imaging device, the multi-level imaging device includes L scanning-level imaging apparatuses, and L≥2.

The adjustment module may further include a second adjustment sub-module and a first unification sub-module.

The second adjustment sub-module is used to adjust an apparatus imaging parameter of each of the L scanning-level imaging apparatuses based on difference information between the second detection projection information obtained by scanning the calibration phantom with each of the L scanning-level imaging apparatuses and the first detection projection information respectively corresponding to each of the L scanning-level imaging apparatuses, so as to obtain a calibrated target apparatus imaging parameter of each of the L scanning-level imaging apparatuses.

The first unification sub-module is used to perform a coordinate system unification on the target apparatus imaging parameter of each of the L scanning-level imaging apparatuses, so as to obtain the calibrated target parameter.

According to the embodiments of the present disclosure, the calibration phantom is provided in N different calibration regions, and N≥2.

The calibration apparatus further includes a first scanning module and a first determination module.

The first scanning module is used to scan the calibration phantom in each of the N calibration regions by using the imaging device, so as to obtain second detection projection information corresponding to each of the N calibration regions.

The first determination module is used to determine the second detection projection information according to the candidate second detection projection information corresponding to each of the N calibration regions.

According to the embodiments of the present disclosure, the calibration phantom is provided in a calibration region, the calibration phantom provided in the calibration region includes M calibration postures.

The calibration apparatus further includes a second scanning module and a second determination module.

The second scanning module is used to scan the calibration phantom with each of the M calibration postures by using the imaging device, so as to obtain second detection projection information of the calibration phantom corresponding to each of the M calibration postures.

The second determination module is used to determine the second detection projection information according to the candidate second detection projection information corresponding to each of the M calibration postures.

According to the embodiments of the present disclosure, the calibration phantom is provided in a scanning region of the imaging device; and a coverage area of the calibration phantom covering the scanning region is greater than or equal to a preset coverage threshold, where the preset coverage threshold is determined based on a scanning region area of the scanning region.

According to the embodiments of the present disclosure, the calibration phantom includes a calibration phantom unit, and the calibration phantom unit includes a plurality of calibration wires distributed in an array.

According to the embodiments of the present disclosure, the calibration wire includes at least one of: a cylindrical calibration wire, a cubic calibration wire, a conical calibration wire, or a trapezoidal calibration wire.

According to the embodiments of the present disclosure, the parameter of the imaging device includes: a target point position of the radiation source and a detection position of the detector.

According to the embodiments of the present disclosure, any two or more of the calculation module 710, the determination module 720, and the adjustment module 730 may be combined into one module, or any of them may be split into a plurality of modules. Alternatively, at least some of the functionalities of one or more of these modules may be combined with at least some functionalities of other modules and implemented in one module. According to the embodiments of the present disclosure, at least one of the calculation module 710, the determination module 720 and the adjustment module 730 may be at least partially implemented as hardware circuits, such as field programmable gate array (FPGA), programmable logic array (PLA), system on chip, system on substrate, system on package, application specific integrated circuit (ASIC), or it may be implemented by hardware or firmware in any other reasonable way that integrates or encapsulates the circuit, or it is implemented by any one of software, hardware, and firmware or an appropriate combination thereof. Alternatively, at least one of the calculation module 710, the determination module 720 and the adjustment module 730 may be at least partially implemented as a computer program module, and when the computer program module is executed, the corresponding function may be performed.

Based on the calibration method and/or calibration apparatus for the imaging device described above, the embodiments of the present disclosure further provide an imaging device. The device will be described in detail below in conjunction with FIG. 8.

FIG. 8 schematically shows an application scenario of an imaging device according to an embodiment of the present disclosure.

As shown in FIG. 8, in this embodiment, the imaging device may include a radiation source 810, a conveying apparatus 820, a detector 830 and a calibration apparatus 840.

The radiation source 810 is used to emit a radiation beam.

The conveying apparatus 820 is used to convey a detected object, where a conveying channel is defined above the conveying apparatus, and a calibration phantom is provided in the conveying channel.

The detector 830 is used to receive the radiation beam passing through the detected object or the calibration phantom.

The calibration apparatus 840 may include the calibration apparatus in the above embodiments.

The radiation source 810 and the detector 830 may be used to scan the calibration phantom 830 to obtain the second detection projection information of the calibration phantom 850 at a plurality of calibration positions in the conveying channel, and send the second detection projection information to the calibration apparatus 840.

The calibration apparatus 840 may also be used to calculate the first detection projection information according to the theoretical target point position of the radiation source 810, the theoretical detection position of the detector 830, various calibration positions of the calibration phantom 850, the linear attenuation coefficient of the calibration phantom, and the radiation energy spectrum distribution curve of the radiation source 810.

In this way, the first detection projection information and the second detection projection information may be used to calibrate the imaging device, so that after the calibration is completed, the imaging device may detect and image the detected object moving in the conveying device 820, so as to obtain high-quality detection image information for the detected object.

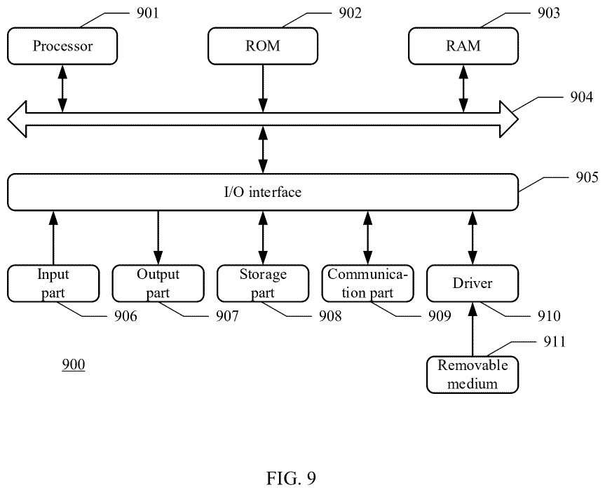

FIG. 9 schematically shows a block diagram of an electronic device suitable for implementing a calibration method for an imaging device according to an embodiment of the present disclosure.

As shown in FIG. 9, the electronic device 900 according to the embodiments of the present disclosure includes a processor 901, which may perform various appropriate actions and processes according to a program stored in a read only memory (ROM) 902 or a program loaded from a storage part 908 into a random access memory (RAM) 903. The processor 901 may include, for example, a general-purpose microprocessor (for example, a CPU), an instruction set processor and/or a related chipset and/or a special-purpose microprocessor (for example, an application specific integrated circuit (ASIC)), etc. The processor 901 may also include an on-board memory for caching purposes. The processor 901 may include a single processing unit or multiple processing units for performing different actions of the method flow according to the embodiments of the present disclosure.

In the RAM 903, various programs and data required for the operation of the electronic device 900 are stored. The processor 901, the ROM 902 and the RAM 903 are connected to each other through a bus 904. The processor 901 performs various operations of the method flow according to the embodiments of the present disclosure by performing programs in the ROM 902 and/or RAM 903. It should be noted that the program may also be stored in one or more memories other than the ROM 902 and the RAM 903. The processor 901 may also perform various operations of the method flow according to the embodiments of the present disclosure by performing programs stored in the one or more memories.

According to the embodiments of the present disclosure, the electronic device 900 may further include an input/output (I/O) interface 905, and the input/output (I/O) interface 905 is also connected to the bus 904. The electronic device 900 may also include one or more of the following components connected to the I/O interface 905: an input part 906 including a keyboard, a mouse, etc.; an output part 907 including such as a cathode ray tube (CRT), a liquid crystal display (LCD), a speaker, etc.; a storage part 908 including a hard disk, etc.; and a communication part 909 including a network interface card such as a LAN card, a modem, etc. The communication part 909 performs communication processing through a network such as the Internet. A driver 910 is also connected to the I/O interface 905 as required. A removable medium 911, such as a magnetic disk, an optical disk, a magneto-optical disk, a semiconductor memory, etc., is installed on the driver 910 as required, so that the computer program read therefrom is installed into the storage part 908 as required.

The present disclosure further provides a computer-readable storage medium. The computer-readable storage medium may be included in the device/apparatus/system described in the above mentioned embodiments; or it may exist alone without being assembled into the apparatus/device/system. The above mentioned computer-readable storage medium carries one or more programs, and when the above mentioned one or more programs are executed, the method according to the embodiments of the present disclosure is implemented.

According to the embodiments of the present disclosure, the computer-readable storage medium may be a non-volatile computer-readable storage medium, for example, may include but not limited to: portable computer disk, hard disk, random access memory (RAM), read-only memory (ROM), erasable programmable read-only memory (EPROM or flash memory), portable compact disk read-only memory (CD-ROM), optical storage device, magnetic storage device, or any suitable combination of the above. In the present disclosure, the computer-readable storage medium may be any tangible medium that contains or stores a program, and the program may be used by or in combination with an instruction execution system, apparatus, or device. For example, according to the embodiments of the present disclosure, the computer-readable storage medium may include the ROM 902 and/or RAM 903 and/or one or more memories other than ROM 902 and RAM 903 described above.

The embodiments of the present disclosure further include a computer program product, which includes a computer program. The computer program contains program codes for implementing the method shown in the flowchart. When the computer program product is run in a computer system, the program codes are used to enable the computer system to implement the calibration method provided in the embodiments of the present disclosure.

When the computer program is executed by the processor 901, it executes the above mentioned functions defined in the system/apparatus of the embodiments of the present disclosure. According to the embodiments of the present disclosure, the systems, apparatuses, modules, units, etc. described above may be implemented by computer program modules.

In one embodiment, the computer program may rely on tangible storage mediums such as optical storage devices, magnetic storage devices, etc. In another embodiment, the computer program may also be transmitted and distributed in the form of signals on the network medium, and downloaded and installed through the communication part 909, and/or installed from the removable medium 911. The program codes contained in the computer program may be transmitted via any suitable network medium, including but not limited to wireless, wired, etc., or any suitable combination thereof.

In such the embodiments, the computer program may be downloaded and installed from the network through the communication part 909, and/or installed from the removable medium 911. When the computer program is executed by the processor 901, it executes the above mentioned functions defined in the system of the embodiments of the present disclosure. According to the embodiments of the present disclosure, the systems, devices, apparatuses, modules, units, etc. described above may be implemented by computer program modules.

According to the embodiments of the present disclosure, program codes for executing the computer program provided by the embodiments of the present disclosure may be written in any combination of one or more programming languages. Specifically, these computing programs may be implemented using high-level procedural and/or object-oriented programming languages, and/or assembly/machine languages. The programming languages include but are not limited to Java, C++, Python, “C” language, or similar programming languages. The program codes may be executed entirely on user computing devices, partially on user devices, partially on remote computing devices, or entirely on remote computing devices or servers. In cases involving the remote computing devices, the remote computing devices may be connected to the user computing devices through any type of network, including local area networks (LANs) or wide area networks (WANs), or may be connected to external computing devices (such as using Internet service providers to connect via the Internet).

The flowcharts and block diagrams in the accompanying drawings illustrate the possible architectures, functions, and operations implemented by the system, the method, and the computer program product according to various embodiments of the present disclosure. In this regard, each box in the flowchart or the block diagram may represent a module, a program segment, or a part of code that contains one or more executable instructions for implementing specified logical functions. It should also be noted that in some alternative implementations, the functions marked in the boxes may be executed in a different order than those marked in the accompanying drawings. For example, two consecutive boxes may actually be executed in parallel, and sometimes they may also be executed in a reverse order, depending on the functions involved. It should also be noted that each box in the block diagram or the flowchart, as well as combinations of boxes in the block diagram or the flowchart, may be implemented using dedicated hardware-based systems that perform specified functions or operations, or may be implemented using a combination of dedicated hardware and computer instructions.

Those skilled in the art may understand that the features described in the various embodiments of the present disclosure and/or the claims may be combined and/or incorporated in various ways, even if such combinations or incorporations are not explicitly described in the present disclosure. In particular, without departing from the spirit and teachings of the present disclosure, the various embodiments of the present disclosure and/or the features described in the claims may be combined and/or incorporated in various ways. All these combinations and/or incorporations fall within the scope of the present disclosure.

The embodiments of the present disclosure have been described above. However, these embodiments are for illustrative purposes only, and are not intended to limit the scope of the present disclosure. Although the respective embodiments are described above, this does not mean that the measures in the respective embodiments may not be advantageously used in combination. The scope of the present disclosure is defined by the appended claims and their equivalents. Those skilled in the art may make various substitutions and modifications without departing from the scope of the present disclosure, and these substitutions and modifications should all fall within the scope of the present disclosure.

1. A calibration method for an imaging device, comprising:
calculating a first theoretical intersection line according to a theoretical target point position of a radiation source of the imaging device, a theoretical detection position of a detector, and a phantom position of a calibration phantom, wherein the first theoretical intersection line represents a propagation path formed within the calibration phantom after a connecting line between the theoretical target point position and the theoretical detection position passes through the calibration phantom;
determining first detection projection information for the calibration phantom based on the first theoretical intersection line, an attenuation coefficient of a radiation emitted by the radiation source in the calibration phantom, and a radiation energy spectrum distribution of the radiation source in the imaging device; and
adjusting a parameter of the imaging device by using difference information between the first detection projection information and second detection projection information obtained by scanning the calibration phantom with the imaging device, so as to calibrate the imaging device and obtain a calibrated target parameter.

2. The calibration method according to claim 1, wherein the adjusting a parameter of the imaging device by using difference information between the first detection projection information and second detection projection information obtained by scanning the calibration phantom with the imaging device comprises:
processing the difference information between the first detection projection information and the second detection projection information according to a target function to obtain the target parameter, wherein target difference information between target second detection projection information obtained by scanning the calibration phantom and the first detection projection information converges, wherein the target difference information is obtained after the imaging device is calibrated according to the target parameter.

3. The calibration method according to claim 2, wherein the target function comprises a mapping function and a loss function;
wherein the processing the difference information between the first detection projection information and the second detection projection information according to a target function to obtain the target parameter comprises:
processing the first detection projection information and the second detection projection information by using the mapping function, so as to obtain a first projection mapping value and a second projection mapping value respectively;
processing the first projection mapping value and the second projection mapping value by using the loss function, so as to obtain a loss value;
iteratively adjusting the parameter of the imaging device according to the loss value until the loss function converges; and
determining the parameter of the imaging device as the target parameter when the loss function converges.

4. The calibration method according to claim 1, wherein the imaging device comprises a multi-level imaging device, the multi-level imaging device comprises L scanning-level imaging apparatuses, and L≥2;
wherein the adjusting a parameter of the imaging device by using difference information between the first detection projection information and second detection projection information obtained by scanning the calibration phantom with the imaging device comprises:
adjusting an apparatus imaging parameter of each of the L scanning-level imaging apparatuses based on difference information between the second detection projection information obtained by scanning the calibration phantom with each of the L scanning-level imaging apparatuses and the first detection projection information respectively corresponding to each of the L scanning-level imaging apparatuses, so as to obtain a calibrated target apparatus imaging parameter of each of the L scanning-level imaging apparatuses; and
performing a coordinate system unification on the target apparatus imaging parameter of each of the L scanning-level imaging apparatuses, so as to obtain the calibrated target parameter.

5. The calibration method according to claim 1, wherein the calibration phantom is provided in N different calibration regions, and N≥2;
wherein the calibration method further comprises:
scanning the calibration phantom in each of the N calibration regions by using the imaging device, so as to obtain candidate second detection projection information corresponding to each of the N calibration regions; and
determining the second detection projection information according to the candidate second detection projection information corresponding to each of the N calibration regions.

6. The calibration method according to claim 1, wherein the calibration phantom is provided in a calibration region, the calibration phantom provided in the calibration region comprises M calibration postures, and M≥2;
wherein the calibration method further comprises:
scanning the calibration phantom with each of the M calibration postures by using the imaging device, so as to obtain candidate second detection projection information of the calibration phantom corresponding to each of the M calibration postures; and
determining the second detection projection information according to the candidate second detection projection information corresponding to each of the M calibration postures.

7. The calibration method according to claim 1,
wherein the calibration phantom is provided in a scanning region of the imaging device; and
wherein a coverage area of the calibration phantom covering the scanning region is greater than or equal to a preset coverage threshold, and the preset coverage threshold is determined based on a scanning region area of the scanning region.

8. The calibration method according to claim 1, wherein the calibration phantom comprises a calibration phantom unit, and the calibration phantom unit comprises a plurality of calibration wires distributed in an array.

9. The calibration method according to claim 8, wherein the calibration wire comprises at least one of: a cylindrical calibration wire, a cubic calibration wire, a conical calibration wire, or a trapezoidal calibration wire.

10. The calibration method according to claim 1, wherein the parameter of the imaging device comprises: a target point position of the radiation source and a detection position of the detector.

11. A calibration apparatus for an imaging device, comprising:
a calculation module configured to calculate a first theoretical intersection line according to a theoretical target point position of a radiation source of the imaging device, a theoretical detection position of a detector, and a phantom position of a calibration phantom, wherein the first theoretical intersection line represents a propagation path formed within the calibration phantom after a connecting line between the theoretical target point position and the theoretical detection position passes through the calibration phantom;
a determination module configured to determine first detection projection information for the calibration phantom based on the first theoretical intersection line, an attenuation coefficient of a radiation emitted by the radiation source in the calibration phantom, and a radiation energy spectrum distribution of the radiation source in the imaging device; and
an adjustment module configured to adjust a parameter of the imaging device by using difference information between the first detection projection information and second detection projection information obtained by scanning the calibration phantom with the imaging device, so as to calibrate the imaging device and obtain a calibrated target parameter.

12. The calibration apparatus according to claim 11, wherein the adjustment module is further configured to:
process the difference information between the first detection projection information and the second detection projection information according to a target function to obtain the target parameter, wherein target difference information between target second detection projection information obtained by scanning the calibration phantom and the first detection projection information converges, wherein the target difference information is obtained after the imaging device is calibrated according to the target parameter.

13. The calibration apparatus according to claim 12, wherein the target function comprises a mapping function and a loss function;
wherein processing the difference information between the first detection projection information and the second detection projection information according to the target function to obtain the target parameter comprises:
processing the first detection projection information and the second detection projection information by using the mapping function, so as to obtain a first projection mapping value and a second projection mapping value respectively;
processing the first projection mapping value and the second projection mapping value by using the loss function, so as to obtain a loss value;
iteratively adjusting the parameter of the imaging device according to the loss value until the loss function converges; and
determining the parameter of the imaging device as the target parameter when the loss function converges.

14. The calibration apparatus according to claim 11, wherein the imaging device comprises a multi-level imaging device, the multi-level imaging device comprises L scanning-level imaging apparatuses, and L≥2;
wherein the adjustment module is further configured to:
adjust an apparatus imaging parameter of each of the L scanning-level imaging apparatuses based on difference information between the second detection projection information obtained by scanning the calibration phantom with each of the L scanning-level imaging apparatuses and the first detection projection information respectively corresponding to each of the L scanning-level imaging apparatuses, so as to obtain a calibrated target apparatus imaging parameter of each of the L scanning-level imaging apparatuses; and
perform a coordinate system unification on the target apparatus imaging parameter of each of the L scanning-level imaging apparatuses, so as to obtain the calibrated target parameter.

15. The calibration apparatus according to claim 11, wherein the calibration phantom is provided in N different calibration regions, and N≥2;
wherein the calibration apparatus further comprises:
a first scanning module configured to scan the calibration phantom in each of the N calibration regions by using the imaging device, so as to obtain second detection projection information corresponding to each of the N calibration regions; and
a first determination module configured to determine the second detection projection information according to the candidate second detection projection information corresponding to each of the N calibration regions.

16. The calibration apparatus according to claim 11, wherein the calibration phantom is provided in a calibration region, the calibration phantom provided in the calibration region comprises M calibration postures;
wherein the calibration apparatus further comprises:
a second scanning module configured to scan the calibration phantom with each of the M calibration postures by using the imaging device, so as to obtain second detection projection information of the calibration phantom corresponding to each of the M calibration postures; and
a second determination module configured to determine the second detection projection information according to the candidate second detection projection information corresponding to each of the M calibration postures.

17. The calibration apparatus according to claim 11,
wherein the calibration phantom is provided in a scanning region of the imaging device; and
wherein a coverage area of the calibration phantom covering the scanning region is greater than or equal to a preset coverage threshold, and the preset coverage threshold is determined based on a scanning region area of the scanning region.

18. The calibration apparatus according to claim 11, wherein the calibration phantom comprises a calibration phantom unit, and the calibration phantom unit comprises a plurality of calibration wires distributed in an array.

19. The calibration apparatus according to claim 18, wherein the calibration wire comprises at least one of: a cylindrical calibration wire, a cubic calibration wire, a conical calibration wire, or a trapezoidal calibration wire.

20. (canceled)

21. An imaging device, comprising:
a radiation source configured to emit a radiation beam;
a conveying apparatus configured to convey a detected object, wherein a conveying channel is defined above the conveying apparatus, and a calibration phantom is provided in the conveying channel;
a detector configured to receive the radiation beam passing through the detected object or the calibration phantom; and
the calibration apparatus of claim 11.
